(19) United States
(12) Patent Application Publication
(10) Pub. No.: US 2022/0020500 A1
(43) Pub. Date: Jan. 20, 2022

(54) SENSOR ASSISTED DEPRESSION DETECTION

(71) Applicant: Samsung Electronics Co., Ltd., Gyeonggi-Do (KR)

(72) Inventors: Jawahar Jain, Los Altos, CA (US); Cody Wortham, Mountain View, CA (US); James Young, Menlo Park, CA (US); Sajid Sadi, San Jose, CA (US); Pranav Mistry, Campbell, CA (US)

(21) Appl. No.: 17/490,365

(22) Filed: Sep. 30, 2021

Related U.S. Application Data
(63) Continuation of application No. 15/296,347, filed on Oct. 18, 2016.
(60) Provisional application No. 62/300,038, filed on Feb. 25, 2016.

(51) Int. Cl. G16H 50/30; G16H 50/20; G16H 20/70

(52) U.S. Cl. CPC G16H 50/30; G16H 20/70; G16H 50/20

(57) ABSTRACT

Detecting depression may include generating, using a sensor, sensor data for a user and automatically detecting, using a processor, a marker for depression in the sensor data. Responsive to determining, using the processor, that a condition is satisfied based upon the marker for depression, a survey is presented using a device.

### CROSS-REFERENCE TO RELATED APPLICATIONS

This application claims the benefit of U.S. Provisional Patent Application No. 62/300,038 filed on Feb. 25, 2016, which is fully incorporated herein by reference.

### RESERVATION OF RIGHTS IN COPYRIGHTED MATERIAL

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### TECHNICAL FIELD

This disclosure relates to detecting depression within users and, more particularly, to detecting depression using a sensor assisted technique that facilitates survey administration and/or survey scoring for detection of depression and/or depressive behavior in users.

### BACKGROUND

The American Psychiatric Association (APA) has estimated that depression costs the United States approximately $40 billion yearly. The world-wide cost of depression is much higher. The costs of depression, however, extends well beyond lost economic output and the cost of medical treatment. Depression is also linked with increases in mortality rates, particularly in groups of patients being treated for significant health issues. For example, in the case of patients being treated for exacerbated cardiac disease, depression can increase mortality rates by 400% or more. Despite this finding, depression goes largely undiagnosed.

The Patient Health Questionnaire (PHQ)-2 and PHQ-9 are validated screening tools commonly used for depression. There is significant evidence that the PHQ-2 and the PHQ-9 are accurate for depression screening in adolescents, adults, and older adults. For example, per the APA, the PHQ-2 has a 97 percent sensitivity and 67 percent specificity in adults. The PHQ-9 has a 61 percent sensitivity and 94 percent specificity in adults. If the PHQ-2 is positive for depression, the PHQ-9 is often administered.

Unfortunately, there are issues relating to the use of the PHQ-2 and/or PHQ-9. One issue is that completing these questionnaires requires significant effort from patients. Patients are often resistant to taking the questionnaires, which contributes to the large number of undiagnosed cases of depression. In fact, routine administration of the PHQ-9 during a long rehabilitation program is often thought to be so onerous that the accuracy of the PHQ-9 becomes highly suspect.

Another issue is that that mood recall, as suggested by research, is difficult and error prone. One's current mood tends to color recollection of one's prior moods. Because the PHQ-2 and/or PHQ-9 inquire about a patient's mood over a two-week time span, the questionnaires are susceptible to faulty mood recall on the part of the patient.

### SUMMARY

An embodiment includes a method of detecting depression. The method can include generating, using a sensor, sensor data for a user and automatically detecting, using a processor, a marker for depression from the sensor data. The method can include, responsive to determining, using the processor, that a condition is satisfied based upon the marker for depression, presenting a survey using a device.

Another embodiment includes an apparatus for detecting depression. The system can include a sensor configured to generate sensor data, a memory configured to store the sensor data, and a processor coupled to the memory. The processor is configured to initiate executable operations. The executable operations can include automatically detecting a marker for depression from the sensor data and, responsive to determining that a condition is satisfied based upon the marker for depression, presenting a survey using a device.

A computer program product includes a computer readable storage medium having program code stored thereon. The program code is executable by a processor to perform operations for detecting depression. The operations include generating sensor data for a user, automatically detecting a marker for depression from the sensor data, and, responsive to determining, using the processor, that a condition is satisfied based upon the marker for depression, presenting a survey using a device.

This Summary section is provided merely to introduce certain concepts and not to identify any key or essential features of the claimed subject matter. Many other features and embodiments of the invention will be apparent from the accompanying drawings and from the following detailed description.

### BRIEF DESCRIPTION OF THE DRAWINGS

The accompanying drawings show one or more embodiments; however, the accompanying drawings should not be taken to limit the invention to only the embodiments shown. Various aspects and advantages will become apparent upon review of the following detailed description and upon reference to the drawings.

FIG. 1 is an example architecture for a device.

FIG. 2 is an example user interface for presenting a survey.

FIG. 3 is an example method of sensor assisted depression detection.

FIG. 4 is another example method of sensor assisted depression detection.

### DETAILED DESCRIPTION

While the disclosure concludes with claims defining novel features, it is believed that the various features described herein will be better understood from a consideration of the description in conjunction with the drawings. The process(es), machine(s), manufacture(s) and any variations thereof described within this disclosure are provided for purposes of illustration. Any specific structural and functional details described are not to be interpreted as limiting, but merely as a basis for the claims and as a representative basis for teaching one skilled in the art to variously employ the features described in virtually any appropriately detailed structure. Further, the terms and phrases used within this disclosure are not intended to be limiting, but rather to provide an understandable description of the features described.

This disclosure relates to detecting depression within users and, more particularly, to detecting depression using a sensor assisted technique that facilitates survey delivery and/or survey scoring for detection of depression and/or depressive behavior in users. One or more example embodiments described herein relate to a device equipped with one or more sensors, methods of operation of the device, and computer readable storage media. The sensors are configured to generate sensor data. The device is capable of analyzing the sensor data to identify one or more markers for depression. The device is capable of determining, based upon the marker(s) for depression, whether a first condition is satisfied.

In response to determining that the first condition is satisfied, the device is capable of presenting a survey (e.g., a first survey). In one example, the survey is designed to indicate the likelihood that the user taking the survey is suffering from depression. The device is capable of receiving survey data from the user in response to the survey. The device is capable of scoring the survey using the survey data. The score provides an indication as to whether the user should seek professional help.

In another embodiment, the device is capable of determining whether a second condition is met. The second condition may include determining that a score for the first survey, based upon received survey data, exceeds a threshold score. If so, the device is capable of utilizing an additional (e.g., a second) survey. In one example, the device is capable of estimating a score for the second survey based, at least in part, upon the sensor data. The device need not present any additional questions from the second survey. In another example, the device is capable of presenting one or more questions, e.g., a subset of questions, from the second survey. The device is capable of utilizing survey data received in response to the second survey in combination with sensor data to estimate a score for the second survey.

One or more of the embodiments described within this disclosure may be used or incorporated into various rehabilitation programs. Depression is one of the problems that limits the gains of therapy for recovering patients. Within this disclosure, rehabilitation in a cardiac program setting is used for purposes of illustration. It should be appreciated, however, that the various embodiments described herein may be applied to any type of rehabilitation program.

In this context, consider that in cardiac rehabilitation, depression not only limits the gains a patient can derive but also may significantly increase the mortality and the morbidity associated with initial cardiac events. Unfortunately, due to resource shortfalls, clinicians may not be well positioned to extensively investigate the mental state of each patient within a rehabilitation program. Hence, determining the depression profile of a patient, and subsequent targeted treatment to the effected subgroups, can be life-saving.

In view of the effectiveness of the Personal Health Questionnaire (PHQ)-2 and the PHQ-9 approaches, if scores for the PHQ-2 and/or PHQ-9 for a given patient can be accurately determined or approximated, then the limited medical resources can be targeted to a high risk subgroup. Accordingly, one or more embodiments described herein are directed to determining the PHQ-2 and the PHQ-9 scores, or assisting in the evaluation of the scores thereby requiring a lesser cognitive load on the part of the patent. In some embodiments, the scores may be estimated or approximated. One or more other embodiments are directed to alerting medical care providers of the need of a more detailed examination for a user.

Further aspects and details of the inventive arrangements are described below with reference to the figures. For purposes of simplicity and clarity of illustration, elements shown in the figures have not necessarily been drawn to scale. For example, the dimensions of some of the elements may be exaggerated relative to other elements for clarity. Further, where considered appropriate, reference numbers are repeated among the figures to indicate corresponding, analogous, or like features.

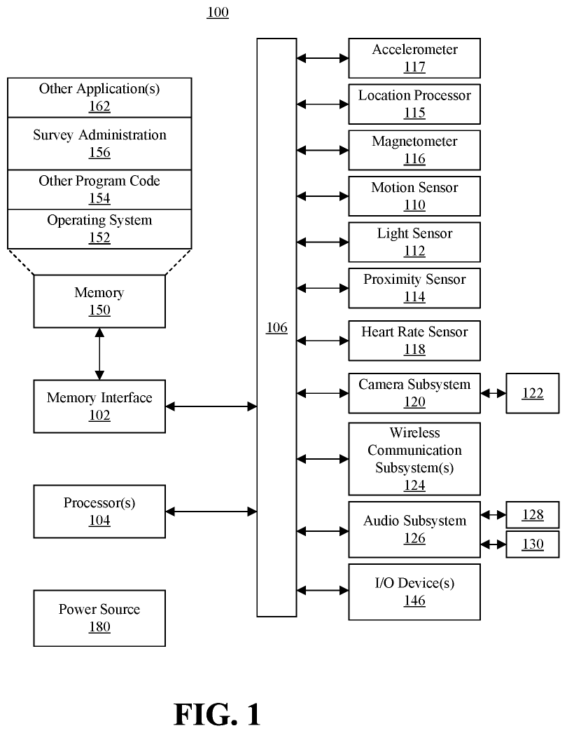

FIG. 1 is an example architecture 100 for a device. In one embodiment, architecture 100 is for a data processing system. Architecture 100 can include a memory interface 102, one or more processors 104 (e.g., image processors, digital signal processors, data processors, etc.), and an interface 106. Memory interface 102, one or more processors 104, and/or interface 106 can be separate components or can be integrated in one or more integrated circuits. The various components in architecture 100, for example, can be coupled by one or more communication buses or signal lines (e.g., interconnects and/or wires).

Sensors, devices, subsystems, and input/output (I/O) devices can be coupled to interface 106 to facilitate the functions and/or operations described herein including the generation of sensor data. The various sensors, devices, subsystems, and/or I/O devices may be coupled to interface 106 directly or through one or more intervening I/O controllers (not shown).

For example, motion sensor 110, light sensor 112, and proximity sensor 114 can be coupled to interface 106 to facilitate orientation, lighting, and proximity functions of a device using architecture 100. Location processor 115 (e.g., a GPS receiver) can be connected to peripherals interface 106 to provide geo-positioning. Electronic magnetometer 116 (e.g., an integrated circuit chip) can also be connected to peripherals interface 106 to provide data that can be used to determine the direction of magnetic North. Thus, electronic magnetometer 116 can be used as an electronic compass. Accelerometer 117 can also be connected to peripherals interface 106 to provide data that can be used to determine change of speed and direction of movement of a device using architecture 100. Heart rate sensor 118 can be connected to peripherals interface 106 to facilitate measurement of a heartbeat and the determination of a heart rate.

Camera subsystem 120 and an optical sensor 122, e.g., a charged coupled device (CCD) or a complementary metal-oxide semiconductor (CMOS) optical sensor, can be utilized to facilitate camera functions, such as recording images and video clips (hereafter “image data”).

Communication functions can be facilitated through one or more wireless communication subsystems 124, which can include radio frequency receivers and transmitters and/or optical (e.g., infrared) receivers and transmitters. The specific design and implementation of communication subsystem 124 can depend on the communication network(s) over which a device using architecture 100 is intended to operate. For example, communication subsystem(s) 124 may be designed to operate over a GSM network, a GPRS network, an EDGE network, a WiFi or WiMax network, a Bluetooth network, and/or any combination of the foregoing. Wireless communication subsystem(s) 124 can include hosting protocols such that a device using architecture 100 can be configured as a base station for other wireless devices.

Audio subsystem 126 can be coupled to a speaker 128 and a microphone 130 to facilitate voice-enabled functions, such as voice recognition, voice replication, digital recording, and telephony functions. Audio subsystem 126 is capable of generating audio type sensor data.

I/O devices 146 can be coupled to interface 106. Examples of I/O devices 146 can include, but are not limited to, display devices, touch sensitive display devices, track pads, keyboards, pointing devices, communication ports (e.g., USB ports), buttons or other physical controls, and so forth. A touch sensitive device such as a display screen and/or a pad is configured to detect contact, movement, breaks in contact, etc., using any of a variety of touch sensitivity technologies. Example touch sensitive technologies include, but are not limited to, capacitive, resistive, infrared, and surface acoustic wave technologies, as well as other proximity sensor arrays or other elements for determining one or more points of contact with a touch sensitive device. One or more of I/O devices 146 may be adapted to control functions of sensors, subsystems, and such of architecture 100.

Architecture 100 further includes a power source 180. Power source 180 is capable of providing electrical power to the various elements of architecture 100. In one embodiment, power source 180 is implemented as one or more batteries. The batteries may be implemented using any of a variety of different battery technologies whether disposable (e.g., replaceable) or rechargeable. In another embodiment, power source 180 is configured to obtain electrical power from an external source and provide power (e.g., DC power) to the elements of architecture 100. In the case of a rechargeable battery, power source 180 further may include circuitry that is capable of charging the battery or batteries.

Memory interface 102 can be coupled to memory 150. Memory 150 can include high-speed random access memory (e.g., volatile memory) and/or non-volatile memory, such as one or more magnetic disk storage devices, one or more optical storage devices, and/or flash memory (e.g., NAND, NOR). Memory 150 can store operating system 152, such as LINUX, UNIX, a mobile operating system, an embedded operating system, etc. Operating system 152 may include instructions for handling basic system services and for performing hardware dependent tasks. In some implementations, operating system 152 can include a kernel.

Memory 150 may also store other program code 154 such as communication instructions to facilitate communicating with one or more additional devices, one or more computers and/or one or more servers; graphical user interface instructions to facilitate graphic user interface processing; sensor processing instructions to facilitate sensor-related processing and functions; phone instructions to facilitate phone-related processes and functions; electronic messaging instructions to facilitate electronic-messaging related processes and functions; Web browsing instructions to facilitate Web browsing-related processes and functions; media processing instructions to facilitate media processing-related processes and functions; GPS/Navigation instructions to facilitate GPS and navigation-related processes and functions; and camera instructions to facilitate camera-related processes and functions. Memory 150 may also store one or more other application(s) 162. Other program code (not shown) may be included to facilitate security functions, web video functions, and so forth.

Memory 150 may store survey administration program code 156 to facilitate analysis of sensor data collected from sensors included in architecture 100, delivery of survey(s) and/or subsets of questions of survey(s) to a user via a device using architecture 100, and/or the scoring of the survey(s). In one embodiment, survey administration program code 156 facilitates estimation (e.g., approximation) of a score for a survey using survey data, sensor data, or a combination of survey data and sensor data. In a further embodiment, survey administration program code 156 facilitates delivery of scores to one or more other systems and/or entities.

Each of the above identified instructions and applications can correspond to a set of instructions for performing one or more functions described above. These instructions may or may not implemented as separate software programs, procedures, or modules. Memory 150 can include additional instructions or fewer instructions. Furthermore, various functions of architecture 100 may be implemented in hardware and/or in software, including in one or more signal processing and/or application specific integrated circuits.

Program code stored within memory 150 and any data items used, generated, and/or operated upon by a device utilizing an architecture the same as or similar to that of architecture 100 are functional data structures that impart functionality when employed as part of the device. Examples of functional data structures include, but are not limited to, sensor data, survey data, marker(s), and so forth. As defined within this disclosure, a “data structure” is a physical implementation of a data model's organization of data within a physical memory. As such, a data structure is formed of specific electrical or magnetic structural elements in a memory. A data structure imposes physical organization on the data stored in the memory as used by an application program executed using a processor.

In one or more other embodiments, one or more of the various sensors and/or subsystems described with reference to architecture 100 may be separate devices that are coupled or communicatively linked to architecture 100 through wired or wireless connections. For example, one or more or all of accelerometer 117, location processor 115, magnetometer 116, motion sensor 110, light sensor 112, proximity sensor 114, camera subsystem 120, audio subsystem, heart rate sensor 118, and so forth may be implemented as separate systems or subsystems that couple to processor 104, memory interface 102, and/or peripherals interface 106 via a wired or wireless connection(s).

Architecture 100 may include fewer components than shown or additional components not illustrated in FIG. 1 depending upon the particular type of device that is implemented. In addition, the particular operating system and/or application(s) and/or other program code included may also vary according to device type. Further, one or more of the illustrative components may be incorporated into, or otherwise form a portion of, another component. For example, a processor may include at least some memory.

While a device configured to perform the operations described herein may utilize architecture 100, architecture 100 is provided for purposes of illustration and not limitation. A device configured to perform the operations described herein may have a different architecture than illustrated in FIG. 1. The architecture may be a simplified version of architecture 100 and include a processor, memory storing instructions, and one or more sensors. The sensors can include any suitable sensor for measuring or determining, acceleration, location, ambient light, speech, sleep, autonomic nervous system (ANS) arousal, heart rate variability (HRV), heart rate (HR), Limbic-Hypothalamic-Pituitary-Adrenal (LHPA) axis activation, emotional valence, or other biological or activity measurements as described in greater detail below.

Examples of devices that may utilize architecture 100 or another computing architecture as described may include, but are to limited to, a smart phone or other mobile device, a wearable computing device (e.g., smart watch, fitness tracker, patch, etc.), a dedicated medical device, a computer (e.g., desktop, laptop, tablet computer, etc.), and any suitable electronic device capable of sensing and processing the sensor data. Furthermore, it will be appreciated that embodiments can be deployed as a standalone device or deployed by multiple devices in distributed client-server networked system.

Table 1 illustrates the PHQ-2. The PHQ-2 is often used for purposes of screening individuals for depression. The PHQ-2 includes two questions relating to the mood of the user over the past two weeks. The answer given by the user has a score of 0, 1, 2, or 3. The PHQ-2 is scored by summing the score for the two questions.

TABLE 1

| Over the past two weeks, | | | | |
|---|---|---|---|---|
| how often have you been | | | More | Nearly |
| bothered by any of the | Not | Several | than half | every |
| following problems? | at all | days | the days | day |
| 1. Little interest or pleasure | 0 | 1 | 2 | 3 |
| in doing things | | | | |
| 2. Feeling down, depressed, | 0 | 1 | 2 | 3 |
| or hopeless | | | | |

Table 2 below illustrates the probability of a user having a major depressive disorder or any depressive disorder based upon possible scores of 1, 2, 3, 4, 5, or 6.

TABLE 2

| | Probability of Major | Probability of any |
|---|---|---|
| PHQ-2 | Depressive Disorder | Depressive Disorder |
| Score | (%) | (%) |
| 1 | 15.4 | 36.9 |
| 2 | 21.1 | 48.3 |
| 3 | 38.4 | 75.0 |
| 4 | 45.5 | 81.2 |
| 5 | 56.4 | 84.6 |
| 6 | 78.6 | 92.9 |

The PHQ-2 does not have significant resolution for elucidating different aspects of depressive behavior. The PHQ-9 is considered more effective in this regard. Table 3 below illustrates the PHQ-9.

TABLE 3?

| | | | More | Nearly |
|---|---|---|---|---|
| Over the past two weeks, how often have you | Not | Several | than half | every |
| been bothered by any of the following problems? | at all | days | the days | day |
| 1. Little interest or pleasure in doing things | 0 | 1 | 2 | 3 |
| 2. Feeling down, depressed, or hopeless | 0 | 1 | 2 | 3 |
| 3. Trouble falling or staying asleep, or | 0 | 1 | 2 | 3 |
| sleeping too much | | | | |
| 4. Feeling tired or having little energy | 0 | 1 | 2 | 3 |
| 5. Poor appetite or overeating | 0 | 1 | 2 | 3 |
| 6. Feeling bad about yourself-or that you are a failure or | 0 | 1 | 2 | 3 |
| have let yourself or your family down | | | | |
| 7. Trouble concentrating on things, such as reading the | 0 | 1 | 2 | 3 |
| newspaper or watching television | | | | |
| 8. Moving or speaking so slowly that other people could | 0 | 1 | 2 | 3 |
| have noticed. Or the opposite-being fidgety or restless that | | | | |
| you have been moving around a lot more than usual | | | | |
| 9. Thoughts that you would be better off dead, or of hurting | 0 | 1 | 2 | 3 |
| yourself in some way | | | | |

Table 4 below shows how the PHQ-9 is scored.

TABLE 4

| PHQ-9 Score | Depression Measure |
|---|---|
| 1-4 | Minimal depression |
| 5-9 | Mild depression |
| 10-14 | Moderate depression |
| 15-19 | Moderately severe depression |
| 20-27 | Severe depression |

A device using architecture 100 or an architecture similar thereto is capable of collecting data using the various sensors of the device or sensors coupled thereto. Within this disclosure, data generated by a sensor is called “sensor data.” The device further is capable of analyzing the sensor data to identify or detect one or more markers for depression.

The baselines used for detection of markers of depression may be determined using any of a variety of different techniques. In one embodiment, the baselines may be generalized across a particular population of users. For example, the baselines may have a resolution along an axis of gender, age, socioeconomic conditions, comorbidity, etc. In that case, such baselines are not specific to the user of the device.

In another embodiment, one or more or all of the baselines used may be specific to the user of the device. For example, such baselines may be determined by analyzing the sensor data of the user during times that the user is not experiencing a depressive mood. In a further embodiment, the determination of whether a marker is detected is based upon baselines adapted for evaluation on a daily basis. For example, the baseline may be one that is adjusted for evaluating sensor data for the current day as opposed to evaluating sensor data over a plurality, e.g., 14, days.

The device is capable of selectively administering one or more surveys based upon monitoring a user for one or more of the markers of depression. Within this disclosure, the term “survey” is used interchangeably with the term “questionnaire.” In one example, the survey is the PHQ-2 or a derivative thereof. In another example, the survey is the PHQ-9 or a derivative thereof.

The following describes various markers for depression and the detection of such markers. A device as described herein is capable of analyzing sensor data to detect the markers discussed. One example marker for depression is a low activity level of the user. The device is capable of determining the activity level of the user using sensor data generated by the accelerometer and/or the motion sensor. The device is capable of comparing the activity level of the user with a baseline activity level. Responsive to determining that the activity level of the user remains below the baseline activity level for at least a minimum amount of time, for example, the device detects the low activity level marker.

In one or more embodiments, the device is capable of classifying activities of the user. The classification may be performed using known machine learning technologies. For example, the device is capable of classifying activities, e.g., daily chores requiring less, etc. compared to other more active activities such as exercise. The device is capable of detecting a lack of variety in the activities. For example, the device is capable of detecting that the user performs bare minimum daily chores. The lack of variety in activities is another way of detecting the low activity marker indicating that the user is engaged in a depressive pattern. In one or more other embodiments, the device is capable of using both activity level in combination with activity classification in detecting the low activity level marker.

Another example marker for depression is reduced amount of time spent outdoors (e.g., or too much time indoors). The device is capable of determining whether the user is outdoors (or indoors) from location data generated by the GPS receiver. The device is capable of determining the amount of time that the user is indoors and/or outdoors and comparing the amount of time outdoors with a baseline amount of time. Responsive to determining that the amount of time spent outdoors by the user does not exceed the baseline amount of time, the device detects the marker of spending reduced time outdoors.

Another example marker for depression is being homebound. The device is capable of determining whether the user is homebound (e.g., at home or at a particular location) using location data and comparing the amount of time spend at the designated location to a baseline amount of time. Responsive to determining that the amount of time spent at the designated location exceeds the baseline amount of time, the device detects the homebound marker.

Another example marker for depression is a low level of interaction with other people. Individuals that are depressed tend to spend less time interacting with others and the outside world. Such individuals tend to exhibit an introverted profile, which can significantly reduce the amount of emotional support the individuals may receive at the particular time that emotional support is most needed.

One form of interaction is speaking with other users. In one embodiment, the device is capable of using audio data to determine an amount of time that the user is interacting with other persons. The device is capable of sampling audio using the microphone from time-to-time throughout the day, periodically, or responsive to particular events. For example, the device is capable of sampling audio using the microphone when the user may be engaging in a face to face conversation. The device is capable of analyzing the audio, e.g., performing voice analysis and/or voice recognition, to determine whether the user is speaking and/or speaking with another person. Further, the device is capable of measuring the amount of time spent speaking based upon the analysis. The device further may approximate the amount of time spent speaking based upon the frequency at which samples are acquired and/or the number of samples acquired.

In another embodiment, the device is capable of analyzing call logs, which are considered part of the sensor data for purposes of this disclosure, to determine the amount of time the user spent talking with others. The device is capable of determining the total amount of time using one or both of the techniques described. For example, the device may sum the time spent speaking as determined from the call logs and the sampled audio data.

In one or more other embodiments, the device is capable of determining the amount of time spent, e.g., via call logs, interacting with others through voluntary conversation with friends and/or family members. The party to which the user is speaking and the party's relationship to the user may be determined, for example, from a contact list stored within the device or a contact list that is accessible by the device. The device is capable of using the relationship of the other party on a call as an indicator of the user's level of enthusiasm in interacting with the external world. Lack of enthusiasm is marker of well-known energy dynamics involved in personal interaction with the external world and an indicator of a melancholy mood.

The device is capable of comparing the amount of time spent interacting with other persons with a baseline amount of time for interacting with other persons. The device is further capable of determining a measure of enthusiasm and comparing the level of enthusiasm with an energy dynamics baseline. Responsive to determining that the amount of time spent interacting with other persons does not exceed the baseline amount of time for interacting with other persons and/or that the user's level of enthusiasm is below the energy dynamics baseline, the device detects the low level of interaction marker. In one or more other embodiments, the user's relationship to the other party on a call may be used as a quality factor, e.g., a multiplier, for the call time with that user to weight calls with family or friends more heavily than other calls. Similarly, calls determined to be with persons other than family and/or friends, e.g., business calls and/or telemarketing calls, may be unweighted (have a quality factor of 1) or weighted using a quality factor less than one for purposes of comparison to a baseline. In this manner, calls may be valued differently for purposes of comparison with a baseline based upon the relationship of the party to whom the user is talking.

In another embodiment, the device is capable of analyzing the tone and/or modulation of the user's voice as a marker for depression. The tone and/or modulation of the user's voice is an indicator of mood of the user. The device, for example, is capable of detecting crying, supplicatory speech, apathic (disinterested) syndrome, length in time of pauses, (average) vocal pitch, mean loudness, and/or variation of loudness over time. Responsive to determining one or more of the characteristics of the user's voice noted herein, the device detects a marker of depression. The marker for depression may be an independent marker for depression or a subset of the low level of interaction marker.

Another example marker for depression is decreased sleep. Users with depression may be prone to insomnia or disturbed sleep which can be determined using one or more sensors. For example, the device is capable of measuring sleep of the user using HR data and accelerometer data. The device is capable of determining the amount of time that the user sleeps and comparing the amount of time spent sleeping with a baseline amount of time. Responsive to determining that the amount of time the user sleeps does not exceed a baseline amount of time for sleep, the device detects the decreased sleep marker. Another sign of worsening psychophysiological resilience can be detected during sleep via the measurement of HR or blood pressure (BP) as during sleep a person often has a much lesser extent of dipping phenomenon (for HR or BP) as compared to healthy individuals.

Another example marker for depression is significant user supine time. The device is capable of using accelerometer data to determine that the user is supine and the amount of time that the user is supine. The device is capable of comparing the amount of time that the user is supine with a baseline supine time. Responsive to determining that the amount of time that the user is supine exceeds the baseline supine time, the device detects the significant supine time marker.

Another example marker for depression is low ANS arousal. Depression can affect the ANS arousal profile of the user. When under depression the user's ANS arousal and valence are typically in the 3rd quadrant of the Circumplex Model of Emotions, which can be determined by various methods that can detect ANS arousal and valence such as HR and HRV analysis where both trend down at the same time. In one embodiment, the device is capable of using heart rate sensor data to determine HR and/or HRV. For example, the device is capable of determining whether the user is subject to stress and whether the amount of stress exceeds a baseline amount of stress based upon HR (e.g., energy) and HRV (e.g., mood) of the user both being low (e.g., below a baseline for HR and/or a baseline for HRV) at the same time and/or remaining low for at least a minimum amount of time.

Another example marker for depression is high stress especially while interacting with the outside world. In one embodiment, the device is capable of using heart rate sensor data to detect stress by determining HR and/or HRV. For example, the device is capable of determining whether the user is subject to stress and whether the amount of stress exceeds a baseline amount of stress based upon HR (e.g., energy) and HRV (e.g., mood) of the user; with the HR being high (above a certain baseline) and HRV being low (e.g., below a baseline) at the same time and remaining so for at least a minimum amount of time. In another embodiment, the HRV method used may be a sympathovagal balance based HRV method. In one or more other embodiments, the device is capable of performing HRV analysis with the external world by sound analysis. In these embodiments, generally the sound is generated from a live source (as in contrast to a sound coming from an electronic media). A user suffering from depression typically has far more instances of stress arousal when interacting with the outside world. The device is capable of comparing the HRV of the user with a baseline HRV given a same or like sound analysis. Responsive to determining that the HRV of the user matches the baseline, the device detects the ANS arousal marker.

In another embodiment, one may use the GSR (galvanic skin response sensor) of the user to detect the arousal level by itself or with the use of HR, and use the HRV to detect the valence. In general, any method that can detect that valence and/or arousal can be used to determine if the user is located in the 3rd quadrant of Circumplex Model of Emotions. In cases where the user has limited mobility or where there is a robust EEG method, an EEG based approach can also be used which can provide both valence and arousal. One such EEG sensor is the well-known EEG sensor provided by Emotiv of San Francisco, Calif.

In another embodiment, the device includes one or more sensors, e.g., bio-sensors, configured to determine an HRV profile of the user and an amount of chronic stress episodes experienced by the user, which may activate the LHPA axis. Activation of the LHPA axis may be detected by the one or more sensors.

Other example markers include emotional state, etc. In another embodiment, the device is capable of measuring emotional state using image data obtained from the camera and/or facial recognition sensors. The device is capable of analyzing particular features of the user's facial expression within the image data using, for example, the Facial Action Coding Scale (FACS). The device is capable of detecting those facial features indicative of depressive mood (a depressive emotional state). The device, for example, is capable of comparing features found in images over time to determine the amount of time the user spent in a particular mood. Responsive to detecting one or more such facial features and/or determining that the user is in such a state or mood for at least a minimum amount of time, the device detects the emotional state marker.

As discussed, mood recall for a user is often inaccurate. The current mood of the user tends to color or obscure the user's recollection of moods from prior days. In accordance with one or more embodiments described herein, the device is capable of providing questions of the type and/or variety included in the PHQ-2 and/or PHQ-9. The questions may be modified to avoid reference to the past two weeks. For example, the questions may be reformulated to inquire whether the user is currently feeling a particular mood instead of whether the user has experienced such a mood in the past two weeks and how often.

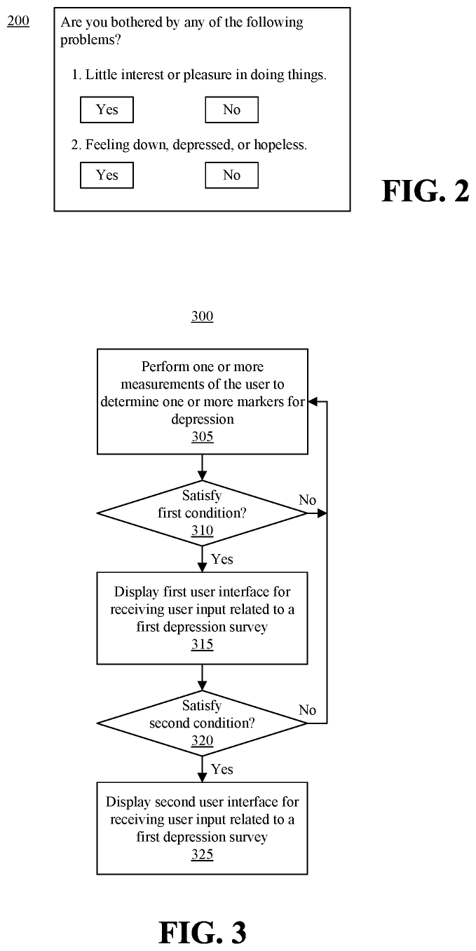

FIG. 2 is an example user interface 200 for presenting a survey. The survey provided in user interface 200 is adapted from the PHQ-2 of Table 1. As pictured, rather than asking the user about mood over the past two weeks, the questions presented ask the user about his or her mood at the present time. As such, rather than selecting from one of four different answers that are weighted differently, the user is provided with the binary choice of either “Yes” or “No” in answer to each question.

In one embodiment, the device, responsive to detecting one or more markers for depression, is capable of presenting PHQ-2 type question(s) without reference to the past two weeks. Such a question-set can be regarded as one member of a two week set (e.g., having 14 such instances). Responses of the user may be stored in a database or other data structure which has the above information categorized so that a long term picture can be obtained by linearly adding the responses of the previous 14 days.

At any given time, e.g., during rehabilitation, the device is capable of determining whether the previous 14-days of response(s), e.g., the survey data, exceed a threshold score. In one example, the threshold score may be set to 2 for high sensitivity. In another example, the threshold score may be set to 4 for high specificity. In another embodiment, the threshold score may be determined based upon the available resources and the criticalness of the user's condition. For low resource or relatively less extreme conditions, higher specificity can be targeted. In a setting with relatively abundant monitoring resources or more critical health conditions, a higher sensitivity can be targeted.

In one embodiment, if the score of the user exceeds the threshold score, the device is capable of presenting the PHQ-9 and/or a derivative thereof. Further analysis of the user's state of mind may be performed based upon the PHQ-9. The PHQ-9 can also be administered in the above manner where only a daily “slice” of the PHQ-9 is presented to the user. The information over two weeks is updated and evaluated as is the case for the PHQ-2. In still another embodiment, if the score exceeds a pre-determined threshold, the device may automatically refer the user to a medical provider. In an alternative embodiment, the survey data may be flagged to a medical provider, so that additional investigation can be conducted as to the mental state of the user as appropriate.

Because users are often resistant to filling out surveys and, in particular, surveys directed to depression, the device is capable of automatically administering one or more surveys. The survey(s) may be administered over one or more days, e.g., within a given time interval. The device administers a survey responsive to determining that a condition is met based upon one or more of the detected markers.

FIG. 3 is an example method 300 of sensor assisted depression detection. Method 300 may be implemented by a device having an architecture the same as, or similar to, the architecture of FIG. 1 or as otherwise described within this disclosure. In one embodiment, the performance of method 300 may be limited or restricted so that the first survey or the second survey is presented no more than one time per day. Further aspects and details are described below with reference to FIG. 3.

In block 305, the device performs one or more measurements of the user to determine one or more of the markers of depression. For example, the device utilizes the sensors to generate and/or collect sensor data. The device further is capable of analyzing the sensor data to detect or identify markers for depression. In identifying or detecting markers for depression, the device is capable of comparing sensor data that is collected with one or more baselines.

In block 310, the device determines whether a first condition is satisfied. Satisfaction of the first condition triggers presentation of the first survey. In one embodiment, the first condition defines the number markers for depression that are to be detected before a first survey is presented to the user. In one example, the device may satisfy the first condition by detecting one marker during a day. In another example, the device may satisfy the condition by detecting two or more different markers during the day. In any case, if the first condition is satisfied, method 300 proceeds to block 315. If the first condition is not satisfied, method 300 can loop back to block 305.

In block 315, the device displays a first user interface for receiving user input related to a first depressive survey. For example, the device displays one or more questions of the variety of the PHQ-2. As noted, the questions may lack reference to the prior two weeks. For example, the device may present a user interface as described in connection with FIG. 2. The device is capable of receiving survey data in the form of responses to the questions from the user via the presented user interface.

In block 320, the device determines whether a second condition is satisfied. If so, method 300 continues to block 325. If not, method 300 loops back to block 305. In one embodiment, the device determines whether the score of the first survey exceeds a threshold score. The threshold score may be one that is indicative of depression in the user.

In block 325, the device displays a second user interface for receiving user input related to a second depressive survey. In one embodiment, the second survey is the PHQ-9 or a derivative thereof. For example, the questions presented by the second user interface may lack reference to a prior time period as is the case with the first user interface and the first survey.

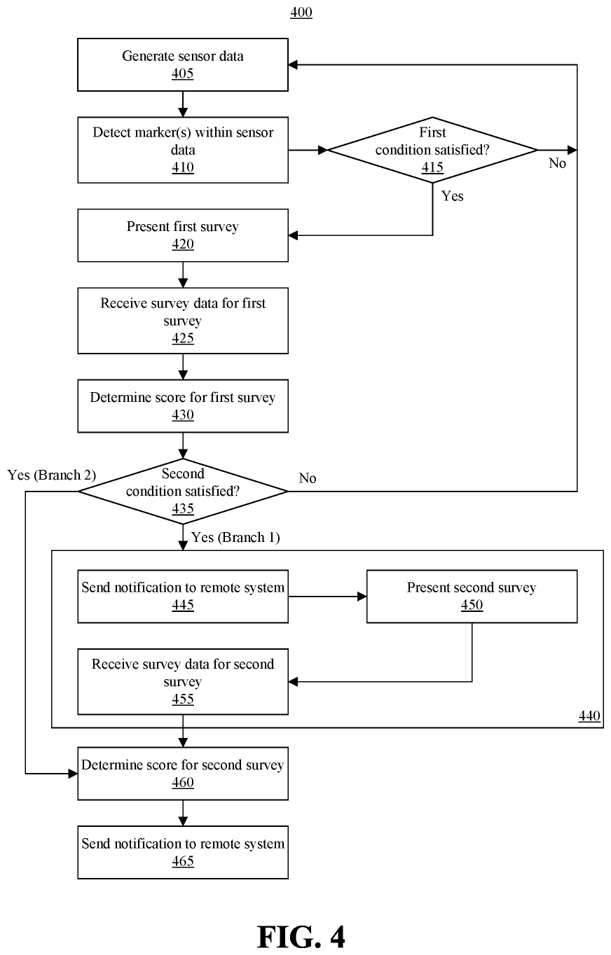

FIG. 4 is an example method 400 of sensor assisted depression detection. Method 400 may be implemented by a device having an architecture the same as, or similar to, the architecture of FIG. 1 or as otherwise described within this disclosure. In one embodiment, the performance of method 400 may be limited or restricted so that the first survey or the second survey is presented no more than one time per day. Further aspects and details are described below with reference to FIG. 4.

In block 405, the device generates sensor data. For example, one or more of the sensors of the device generate sensor data that may be stored in memory of the device as one or more data structures. Examples of sensor data include accelerometer data generated by the accelerometer; location data (e.g., GPS coordinates) generated by the location processor and/or motion sensor; proximity data generated by the proximity sensor; image data generated by the camera subsystem; audio data generated by the audio subsystem; heart rate data generated by the heart rate sensor, and so forth. The device is capable of generating and storing sensor data over a plurality of days.

In block 410, the device is capable of detecting one or more markers within the sensor data. For example, the device is capable of analyzing the sensor data to determine whether one or more markers exist within the sensor data.

In block 415, the device determines whether a first condition is satisfied. Satisfaction of the first condition triggers presentation of the first survey. In one embodiment, the first condition defines the number markers for depression that are to be detected before a first survey is presented to the user. In one example, the device may satisfy the first condition by detecting one marker during a day. In another example, the device may satisfy the condition by detecting two or more different markers during the day. In any case, if the first condition is satisfied, method 400 proceeds to block 420. If the first condition is not satisfied, method 400 can loop back to block 405 to continue generating sensor data and monitoring for marker(s) for depression within the sensor data.

In block 420, the device presents the first survey. The device is capable of presenting the questions of the survey through a user interface of the device. In one embodiment, the device presents the PHQ-2 or an adaptation thereof. As noted, one adaptation is that questions are asked regarding how the user currently feels as opposed to how the user has felt over the past 14 days.

In one example, the device displays the questions of the survey through a visual user interface. For example, the device is capable of displaying a user interface as shown in FIG. 2. While FIG. 2 illustrates both questions being presented concurrently, in another embodiment, the device may present the questions one at a time in serial fashion. In another embodiment, the device may read the questions of the survey aloud to the user. It should be appreciated that the particular modality used to provide the survey through the device is not intended as a limitation of the example embodiments described herein.

In block 425, the device receives survey data for the first survey as specified by one or more received user inputs. The user interface of the device is configured to receive user input providing answers to the questions referred to herein as survey data. The user input may be touch user input, keyboard user input, speech, and so forth. The user input specifying the survey data may be provided using any of a variety of different modalities.

In one embodiment, the device is configured to present the first survey no more than one time within a specific time period. For example, responsive to determining that the first condition is met, the device presents the first survey. The device does not provide the first survey to the user again within the time period regardless of whether the first condition is again met during that same time period. In one example, the time period is a calendar day. In another example, the time period is 24 hours. In order to present the first survey again and obtain further survey data, the device first determines that a new time period has begun and that the first condition is satisfied in the new time period.

The device is further capable of storing received survey data for at least an amount of time necessary to determine a score for the first and/or second surveys. If, for example, the window of time considered for a particular survey is 14 days, the device is capable of storing survey data for at least 14 days. The device may store survey data longer than the required window of time and only utilize the survey data within the window of time when calculating scores for the first and/or second surveys. Appreciably, the device stores the survey data in association with a time and date stamp.

In block 430, the device determines a score for the first survey. In one embodiment, the score is an estimated score. The device determines whether the user provided an affirmative (e.g., a “Yes”) answer to each question of the first survey from the survey data. Table 5 below illustrates how each question of the first survey is scored based upon whether the answer was “No” or “Yes.” The score for each question is summed to determine a score for the first survey. Within Table 5, the value of N is the number of days that the particular question being scored in the first survey is answered affirmatively over a window of time “M.”

TABLE 5

| Answer | Scoring |
|---|---|
| No | 0 |
| Yes (1≤N≤7) | 1+(N−1)/7 |
| Yes (8≤N≤12) | 2+(N−8)/5 |
| Yes (13≤N≤14) | 3 |

For purposes of illustration, consider the case where the user is presented with question 1 and answers affirmatively, e.g., with a “Yes.” Further, the user has answered question 1 of survey 1 affirmatively one other time within the window of time. The time window is 14 days in this example. In that case, the value of N for question 1 is 2. The device calculates the score for question 1 of survey 1 using the expression 1+(N−1)/7 with N=2 to obtain a score for question 1 of 0.286. The device is capable of storing survey data for the window of time. Thus, with the passing of each day, the window of time is a sliding window of time, e.g., a sliding 14 day window in this example.

The device scores the second question in the same way as question 1. It should be appreciated, however, that the value of N for a question is specific to that question and depends upon the number of times that particular question has been answered affirmatively over the window of time. Since the device scores the question 2 using the same technique as question 1, but using a value of N that is specific to question 2, the particular expression used to determine a score for question 2 may differ from the expression used to calculate the score for question 1.

In further illustration, consider the case where the user is presented with question 2 and answers affirmatively, e.g., with a “Yes.” The user has answered question 2 of survey 1 affirmatively 8 other times within the window of time. In that case, the value of N for question 2 is 9. The device calculates the score for question 2 of survey 1 using the expression 2+(N−8)/5 with N=9 to obtain a score for question 2 of 0.6. Again, the device scores the second question using the same technique, where the value of N is determined independently for question 2. As such, in this example, the particular expression used to determine the score for question 2 is different from the expression used to calculate the score for question 1.

In further illustration, consider the case where the user is presented with question 1 and answers affirmatively, e.g., with a “Yes.” The user has answered question 1 of survey 1 affirmatively 11 other times within the window of time. In that case, the value of N for question 1 is 12. The device calculates the score for question 1 of survey 1 to be 3. Again, the device scores question 2 using the same technique, where the value of N is determined independently for question 2.

In one embodiment, the window of time or “M” is set to the amount of time or number of days over which the user mood is to be evaluated. For example, both the PHQ-2 and the PHQ-9, when given in a conventional manner, ask the user to evaluate mood over the prior two-week period. The PHQ-2 and/or the PHQ-9 are given one time using a two week look-back period. In the case of FIG. 4, the first survey is given each day that the first condition is met. The score is calculated for that day using the sliding (or rolling) window of time where N is determined for each question independently for the window of time. The window of time is set to 14 days since the look back period for the PHQ-2 and the PHQ-9 is two weeks.

Accordingly, the scoring performed by the device as described with reference to block 430 is adapted for the case where the user answers the survey using binary answers of yes or no with the survey being administered each day that the first condition is met. The PHQ-2 ordinarily utilizes two questions where the user selects one of four possible answers to each question. Each answer is carries a different score. Because the questions of the first survey are directed to how the user is feeling at the time the survey is administered, the responses are binary and the scoring mechanism described above is used.

The expressions described with reference to Table 5 provide a higher bias for higher numbers for N. In another embodiment, a scaling factor nay be added. In still another embodiment, the expressions of Table 5 used may be non-linear for calculating a score for the questions.

In block 435, the device determines whether a second condition is satisfied. If so, method 400 continues down yes branch 1 or yes branch 2. If not, method 400 loops back to block 405 to continue collecting and analyzing sensor data. In one embodiment, the device determines whether the score of the first survey exceeds a threshold score. The threshold score may be one that is indicative of depression in the user.

Yes branch 1 and yes branch 2 illustrate alternative implementations of method 400. Continuing down yes branch 1, for example, the device may perform one or more optional operations illustrated within block 440. In one embodiment, in block 445, the device optionally sends a notification to a remote system. For example, the device may send a message to the system or device of a health care provider, a medical provider, a mental health professional, etc. The message may indicate the score of the first survey or include other data indicating a need for follow-up with the user. The message may be an electronic mail, a text or instant message, an automated call, or another form of communication. The particular type of message that is sent is not intended as a limitation of the embodiments described herein.

In another embodiment, method 400 may bypass block 445 and proceed from block 435 directly to block 450. In block 450, the device may present a second survey. The second survey may be the PHQ-9 or a derivative thereof. In one aspect, the device presents a subset of the questions of the second survey. Within PHQ-9, questions 1 and 2 are identical to questions 1 and 2 of the PHQ-2. Accordingly, since the first two questions of the PHQ-9 are the two questions already presented to the user as the first survey, questions 1 and 2 of the second survey need not be presented.

For example, the device is capable of presenting one or more of questions 3, 4, 5, 6, 7, 8, and/or 9 of PHQ-9. In one embodiment, as noted, the questions are adapted to inquire about the current mood of the user. In one or more other embodiments, the device is capable of estimating answers to a portion of the questions for the second survey based upon sensor data already collected. For example, the device is capable of estimating answers for questions 3, 4, 6, 7, 8, and/or 9 from the sensor data. In illustration, the device may estimate an answer to question 3 based upon accelerometer data and heart rate data or any other suitable sensor or bio-sensing system that is capable of detecting low-valence and low-arousal state of the user's ANS. The device may estimate an answer to question 4 based upon accelerometer data and/or any data that indicates movement or motion of the user. The device may estimate an answer to question 6 using HR and/or HRV. In one or more embodiments, the HRV method used may be a sympathovagal balance based HRV method. The device may estimate an answer for question 7 based upon activity level of the user. The device may estimate an answer for question 8 based upon audio data, accelerometer data (activity), and/or other motion data such as speed of movement of the user. The device may estimate an answer to question 9 using low valence and low ANS arousal (e.g., as may be indicated by HR and/or HRV).

In another embodiment, the device is capable of estimating answers to one or more of questions 3-9 while presenting at least one of questions 3-9 to the user in order to solicit and obtain survey data for the presented question(s). The device is capable of presenting only one or more selected questions of the second survey for which sensor data is less accurate in estimating answers. In one example, the device is capable of presenting only question 5 to the user to obtain survey data for the second survey. In other examples, the device is capable of presenting only questions 5 and 9, presenting only questions 5 and 6, presenting only questions 5, 6, and 9, and so forth.

In block 455, the device receives survey data for each of the questions of the second survey that are presented.

In block 460, the device determines a score for the second survey. As discussed, the device calculates the score based upon any received survey data for the second survey, the score of the first survey (which is the score of the first question and the second question summed), and/or the estimated answers to questions of the second survey as determined from the sensor data. For any questions of the second survey for which an answer is estimated, it should be appreciated that the device is capable of analyzing sensor data over the window of time, e.g., 14 days, to determine a value for N that is question specific and determine a score for the question using the expressions described with reference to Table 5 or derivatives thereof as described herein. Thus, the value of N may be determined for each question of the second survey independently based upon the number of days within the window of time that the markers indicating an affirmative answer to the question are detected. As noted, in some embodiments, in order to detect a marker, the device may need to detect certain characteristics for a minimum amount of time during a day or whatever time period is used as the evaluation period (e.g., 24 hours).

In the case where method 400 proceeds along yes branch 2 from block 435 directly to block 460, the device is capable of estimating an answer for each of questions 3-9 of the second survey. In one embodiment, the device is capable of estimating an answer to question 5 based upon sensor data. In another embodiment, the device may omit question 5 and adjust the scoring for the second survey accordingly. In any case, the device is capable of determining a score, e.g., an estimated score, for the second survey using only the score of the first survey and the estimated answers to the questions of the second survey as determined from the sensor data.

In block 465, the device optionally sends a notification to a remote system. For example, the device may send a message to the system or device of a health care provider, a medical provider, a mental health professional, etc. The message may indicate the score of the second survey or include other data indicating a need for follow-up with the user. The message may be an electronic mail, a text or instant message, an automated call, or another form of communication. The particular type of message that is sent is not intended as a limitation of the embodiments described herein.

In one or more other embodiments, additional sensors may be incorporated to provide measurements that, if available, may be used with the scores. Care providers may be provided information about markers (e.g., for depression or psychological state in general) as computed by the device and/or such other sensors. For example, ECG, camera, and/or ultrasound are several such sensors to determine the RR-intervals and, hence determine if both HR and HRV trend downward (indicating that the emotion of the user is in the 3rd quadrant of the well-known Circumplex Model of Emotions, which is where depression is located). In one embodiment, the magnitude of HRV and HR changes can be assigned a proportional weight based upon the physiological traits of the given person. For example, an elderly person who is taking beta blockers may not see much elevation in HR when under stress but will find the effect on HRV to remain significantly large. Such information can be programmed in the system by the physician who is aware of what marker of ANS is dampened due to medication or an existing pathology. This information can also be programed using publicly and widely available databases of the FDA approved medicines and their side effect.

In one or more other embodiments, the device is capable of querying the user to measure stress right before sleep and/or measuring the quality of sleep to obtain information about the sleep related portion of PHQ-9, e.g., question 3.

In one or more other embodiments, the device is capable of examining pattern(s) of activities of the user. The device, for example, is capable of detecting a sudden decrease in number of active periods along with a decrease in total activity with concomitant changes in other sensor based markers. The device may use such information to answer vitality related portions of PHQ-9 such as question 4.

In one or more other embodiments, the device may obtain daily weight related measurements. The device is capable of estimating an answer to the portions of PHQ-9 relating to changes in appetite, e.g., question 5.

This disclosure uses the PHQ2 and PHQ9 as example depression screening tools. The examples presented herein, however, are not intended as limitations of the embodiments described. Other depression screening tools may be used in place of the PHQ2 and/or PHQ9. In one or more embodiments, a survey such as the Major Depression Inventory (MDI) may be used as a screening tool. In one or more other embodiments, a survey such as the Web-Based Depression and Anxiety Test (WB-DAT) may be used as a screening tool. In each case, the scoring mechanisms described within this disclosure may be used and/or adapted to such other screening tools. For example, responsive to automatically detecting one or more of the markers for depression described herein, the device is capable of providing one or more of the screening tools (e.g., surveys) to the user.

The terminology used herein is for the purpose of describing particular embodiments only and is not intended to be limiting. Notwithstanding, several definitions that apply throughout this document now will be presented.

As defined herein, the singular forms “a,” “an,” and “the” are intended to include the plural forms as well, unless the context clearly indicates otherwise.

As defined herein, the term “another” means at least a second or more.

As defined herein, the terms “at least one,” “one or more,” and “and/or,” are open-ended expressions that are both conjunctive and disjunctive in operation unless explicitly stated otherwise. For example, each of the expressions “at least one of A, B, and C,” “at least one of A, B, or C,” “one or more of A, B, and C,” “one or more of A, B, or C,” and “A, B, and/or C” means A alone, B alone, C alone, A and B together, A and C together, B and C together, or A, B and C together.

As defined herein, the term “automatically” means without user intervention.

As defined herein, the term “computer readable storage medium” means a storage medium that contains or stores program code for use by or in connection with an instruction execution system, apparatus, or device. As defined herein, a “computer readable storage medium” is not a transitory, propagating signal per se. A computer readable storage medium may be, but is not limited to, an electronic storage device, a magnetic storage device, an optical storage device, an electromagnetic storage device, a semiconductor storage device, or any suitable combination of the foregoing. Memory and/or memory elements, as described herein, are examples of a computer readable storage medium. A non-exhaustive list of more specific examples of a computer readable storage medium may include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, or the like.

As defined herein, the term “coupled” means connected, whether directly without any intervening elements or indirectly with one or more intervening elements, unless otherwise indicated. Two elements may be coupled mechanically, electrically, or communicatively linked through a communication channel, pathway, network, or system.

As defined herein, the terms “includes,” “including,” “comprises,” and/or “comprising,” specify the presence of stated features, integers, steps, operations, elements, and/or components, but do not preclude the presence or addition of one or more other features, integers, steps, operations, elements, components, and/or groups thereof.

As defined herein, the term “if” means “when” or “upon” or “in response to” or “responsive to,” depending upon the context. Thus, the phrase “if it is determined” or “if [a stated condition or event] is detected” may be construed to mean “upon determining” or “in response to determining” or “upon detecting [the stated condition or event]” or “in response to detecting [the stated condition or event]” or “responsive to detecting [the stated condition or event]” depending on the context.

As defined herein, the terms “one embodiment,” “an embodiment,” or similar language mean that a particular feature, structure, or characteristic described in connection with the embodiment is included in at least one embodiment described within this disclosure. Thus, appearances of the phrases “in one embodiment,” “in an embodiment,” and similar language throughout this disclosure may, but do not necessarily, all refer to the same embodiment. The terms “embodiment” and “arrangement” are used interchangeably within this disclosure.

As defined herein, the term “output” means storing in physical memory elements, e.g., devices, writing to display or other peripheral output device, sending or transmitting to another system, exporting, or the like.

As defined herein, the term “plurality” means two or more than two.

As defined herein, the term “processor” means at least one hardware circuit configured to carry out instructions contained in program code. The hardware circuit may be an integrated circuit. Examples of a processor include, but are not limited to, a central processing unit (CPU), an array processor, a vector processor, a digital signal processor (DSP), a field-programmable gate array (FPGA), a programmable logic array (PLA), an application specific integrated circuit (ASIC), programmable logic circuitry, and a controller.

As defined herein, the term “real time” means a level of processing responsiveness that a user or system senses as sufficiently immediate for a particular process or determination to be made, or that enables the processor to keep up with some external process.

As defined herein, the term “responsive to” means responding or reacting readily to an action or event. Thus, if a second action is performed “responsive to” a first action, there is a causal relationship between an occurrence of the first action and an occurrence of the second action. The term “responsive to” indicates the causal relationship.

As defined herein, the term “user” means a human being. The term user and “patient” are used interchangeably within this disclosure from time to time.

The terms first, second, etc. may be used herein to describe various elements. These elements should not be limited by these terms, as these terms are only used to distinguish one element from another unless stated otherwise or the context clearly indicates otherwise.

A computer program product may include a computer readable storage medium (or media) having computer readable program instructions thereon for causing a processor to carry out aspects of the present invention. Within this disclosure, the term “program code” is used interchangeably with the term “computer readable program instructions.” Computer readable program instructions described herein may be downloaded to respective computing/processing devices from a computer readable storage medium or to an external computer or external storage device via a network, for example, the Internet, a LAN, a WAN and/or a wireless network. The network may include copper transmission cables, optical transmission fibers, wireless transmission, routers, firewalls, switches, gateway computers and/or edge devices including edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations for the inventive arrangements described herein may be assembler instructions, instruction-set-architecture (ISA) instructions, machine instructions, machine dependent instructions, microcode, firmware instructions, or either source code or object code written in any combination of one or more programming languages, including an object oriented programming language and/or procedural programming languages. Computer readable program instructions may specify state-setting data. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or a WAN, or the connection may be made to an external computer (for example, through the Internet using an Internet Service Provider). In some cases, electronic circuitry including, for example, programmable logic circuitry, an FPGA, or a PLA may execute the computer readable program instructions by utilizing state information of the computer readable program instructions to personalize the electronic circuitry, in order to perform aspects of the inventive arrangements described herein.

Certain aspects of the inventive arrangements are described herein with reference to flowchart illustrations and/or block diagrams of methods, apparatus (systems), and computer program products. It will be understood that each block of the flowchart illustrations and/or block diagrams, and combinations of blocks in the flowchart illustrations and/or block diagrams, may be implemented by computer readable program instructions, e.g., program code.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. In this way, operatively coupling the processor to program code instructions transforms the machine of the processor into a special-purpose machine for carrying out the instructions of the program code. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the operations specified in the flowchart and/or block diagram block or blocks.

The computer readable program instructions may also be loaded onto a computer, other programmable data processing apparatus, or other device to cause a series of operations to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various aspects of the inventive arrangements. In this regard, each block in the flowchart or block diagrams may represent a module, segment, or portion of instructions, which comprises one or more executable instructions for implementing the specified operations. In some alternative implementations, the operations noted in the blocks may occur out of the order noted in the figures. For example, two blocks shown in succession may be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, may be implemented by special purpose hardware-based systems that perform the specified functions or acts or carry out combinations of special purpose hardware and computer instructions.

The corresponding structures, materials, acts, and equivalents of all means or step plus function elements that may be found in the claims below are intended to include any structure, material, or act for performing the function in combination with other claimed elements as specifically claimed.

The description of the embodiments provided herein is for purposes of illustration and is not intended to be exhaustive or limited to the form and examples disclosed. The terminology used herein was chosen to explain the principles of the inventive arrangements, the practical application or technical improvement over technologies found in the marketplace, and/or to enable others of ordinary skill in the art to understand the embodiments disclosed herein. Modifications and variations may be apparent to those of ordinary skill in the art without departing from the scope and spirit of the described inventive arrangements. Accordingly, reference should be made to the following claims, rather than to the foregoing disclosure, as indicating the scope of such features and implementations.

1-20. (canceled)

21. A method executed by a mobile device, the method comprising:
maintaining, in a memory of the mobile device by a processor of the mobile device, a log of phone calls conducted using the mobile device over a window of time including a plurality of time periods, wherein each time period is a 24 hour time period;
determining, using the processor, an amount of time a user of the mobile device interacts with other persons on a per time period basis based on the log of phone calls;
comparing, using the processor, the amount of time the user of the mobile device interacts with the other persons on the per time period basis with a baseline amount of time;
for each time period in which the amount of time the user of the mobile device speaks with the other persons does not exceed the baseline amount of time, generating, using the processor and at most once during the time period, a graphical user interface (GUI) rendered by a display screen of the mobile device, the GUI including a question set inquiring about a current mood of the user and providing a binary response option for each question of the question set;
for each time period the question set is presented via the GUI, receiving, via the GUI, a user input selecting a binary response option for each question of the question set, wherein the processor stores the binary response option selected in the memory of the mobile device for at least the window of time;
for each question of the question set presented by the GUI, selecting, using the processor, an expression from a plurality of expressions stored in the memory, the selecting performed based on a number of the time periods an affirmative response is received for the question as the binary response option;
determining, using the processor, a total score for the window of time by calculating, for each question of the question set presented, a per question score for the window of time using the selected expression for each respective question based on the affirmative responses received for the question and summing the per question scores;
determining whether the total score exceeds a threshold score; and
in response to determining that the total score exceeds the threshold score, transmitting, via a wireless transmitter of the mobile device, an electronic notification to a remote system of a health care provider, wherein the electronic notification indicates a need to follow-up with the user.

22. The method of claim 21, comprising:
periodically sampling audio detected via a microphone and an audio subsystem of the mobile device;
performing voice recognition on the sampled audio using the processor to detect whether a user of the mobile device is involved in a face-to-face conversation during the periodically sampling throughout the window of time; and
wherein the amount of time the user of the mobile device interacts with other persons is determined based on the log of phone calls and whether the user was determined to be involved in the face-to-face conversation from the periodically sampling.

23. The method of claim 21, comprising:
determining, from the call log, an amount of time spent on calls with one or more selected contacts from a contacts list read by the mobile device; and
applying a scaling factor to the amount of time spent on calls with the one or more selected contacts for the determining the amount of time the user of the mobile device interacts with the other persons.

24. The method of claim 21, wherein:
for each time period of the plurality of time periods in which the amount of time the user of the mobile device speaks with the other persons does not exceed the baseline amount of time, the processor detects a marker; and
prior to generating the GUI including the question set for any time period of the window of time, the processor first detects a plurality of markers corresponding to a minimum required number of markers, wherein each marker is detected based on a comparison of sensor generated data with a corresponding baseline.

25. The method of claim 24, comprising:
detecting a further marker by:
generating, using a microphone and an audio subsystem of the mobile device, audio sensor data of a voice of the user; and
analyzing the voice of the user using the processor to detect an indicator of mood, the indicator of mood including at least one of crying, supplicatory speech, or length of time of pauses.

26. The method of claim 24, comprising:
detecting a further marker by:
generating heart rate sensor data and heart rate variability sensor data using a heartrate sensor of the mobile device;
detecting, using the processor, that heart rate and heart rate variability both trend down at a same time from the heart rate sensor data and heart rate variability data; and
wherein the heart rate sensor is connected to interface circuitry in the mobile device to generate the sensor data and facilitate determination of the heart rate and the heart rate variability.

27. The method of claim 24, comprising:
detecting a further marker by:
generating accelerometer sensor data using an accelerometer of the mobile device; and
determining, using the processor, that an amount of supine time of the user exceeds a baseline amount of supine time from the accelerometer sensor data.

28. The method of claim 24, comprising:
weighting a magnitude of a change in a selected marker of the plurality of markers based on a dampening effect on changes in the selected marker caused by a medication taken by the user.

29. The method of claim 21, comprising:
responsive to the total score exceeding the threshold score, automatically determining a further total score using one or more additional questions by estimating a user response to at least one of the one or more additional questions based only on sensor data, wherein the sensor data includes heartrate and heartrate variability sensor data generated from a heartrate sensor and accelerometer sensor data generated from an accelerometer sensor.

30. A mobile device, comprising:
a memory storing executable program code;
a display screen;
a wireless transmitter;
a processor coupled to the memory, the display screen, and the wireless transmitter, wherein the processor is programmed by executing the program code to initiate operations including:
maintaining, in the memory, a log of phone calls conducted using the mobile device over a window of time including a plurality of time periods, wherein each time period is a 24 hour time period;
determining an amount of time a user of the mobile device interacts with other persons on a per time period basis based on the log of phone calls;
comparing the amount of time the user of the mobile device interacts with the other persons on the per time period basis with a baseline amount of time;
for each time period in which the amount of time the user of the mobile device speaks with the other persons does not exceed the baseline amount of time, generating, at most once during the time period, a graphical user interface (GUI) rendered by the display screen, the GUI including a question set inquiring about a current mood of the user and providing a binary response option for each question of the question set;
for each time period the question set is presented via the GUI, receiving, via the GUI, a user input selecting a binary response option for each question of the question set, wherein the processor stores the binary response option selected in the memory of the mobile device for at least the window of time;
for each question of the question set presented by the GUI, selecting an expression from a plurality of expressions stored in the memory, the selecting performed based on a number of the time periods an affirmative response is received for the question as the binary response option;
determining a total score for the window of time by calculating, for each question of the question set presented, a per question score for the window of time using the selected expression for each respective question based on the affirmative responses received for the question and summing the per question scores;
determining whether the total score exceeds a threshold score; and
in response to determining that the total score exceeds the threshold score, transmitting, via the wireless transmitter, an electronic notification to a remote system of a health care provider, wherein the electronic notification indicates a need to follow-up with the user.

31. The system of claim 30, wherein the mobile device includes a microphone and an audio subsystem, and wherein the processor is programmed to initiate operations comprising:
periodically sampling audio detected via the microphone and the audio subsystem of the mobile device;
performing voice recognition on the sampled audio using the processor to detect whether a user of the mobile device is involved in a face-to-face conversation during the periodically sampling throughout the window of time; and
wherein the amount of time the user of the mobile device interacts with other persons is determined based on the log of phone calls and whether the user was determined to be involved in the face-to-face conversation from the periodically sampling.

32. The system of claim 30, wherein the processor is programmed to initiate operations comprising:
determining, from the call log, an amount of time spent on calls with one or more selected contacts from a contacts list read by the mobile device; and
applying a scaling factor to the amount of time spent on calls with the one or more selected contacts for the determining the amount of time the user of the mobile device interacts with the other persons.

33. The system of claim 30, wherein:
for each time period of the plurality of time periods in which the amount of time the user of the mobile device speaks with the other persons does not exceed the baseline amount of time, the processor detects a marker; and
prior to generating the GUI including the question set for any time period of the window of time, the processor first detects a plurality of markers corresponding to a minimum required number of markers, wherein each marker is detected based on a comparison of sensor generated data with a corresponding baseline.

34. The system of claim 33, wherein the mobile device includes a microphone and an audio subsystem, and wherein the processor is programmed to initiate operations comprising:
detecting a further marker by:
generating, using the microphone and the audio subsystem, audio sensor data of a voice of the user; and
analyzing the voice of the user using the processor to detect an indicator of mood, the indicator of mood including at least one of crying, supplicatory speech, or length of time of pauses.

35. The system of claim 33, wherein the processor is programmed to initiate operations comprising:
detecting a further marker by:
generating heart rate sensor data and heart rate variability sensor data using a heartrate sensor of the mobile device;
detecting, using the processor, that heart rate and heart rate variability both trend down at a same time from the heart rate sensor data and heart rate variability data; and
wherein the heart rate sensor is connected to interface circuitry in the mobile device to generate the sensor data and facilitate determination of the heart rate and the heart rate variability.

36. The system of claim 33, wherein the processor is programmed to initiate operations comprising:
detecting a further marker by:
generating accelerometer sensor data using an accelerometer of the mobile device; and
determining, using the processor, that an amount of supine time of the user exceeds a baseline amount of supine time from the accelerometer sensor data.

37. The system of claim 33, wherein the processor is programmed to initiate operations comprising:
weighting a magnitude of a change in a selected marker of the plurality of markers based on a dampening effect on changes in the selected marker caused by a medication taken by the user.

38. The system of claim 30, wherein the processor is programmed to initiate operations comprising:
responsive to the total score exceeding the threshold score, automatically determining a further total score using one or more additional questions by estimating a user response to at least one of the one or more additional questions based only on sensor data, wherein the sensor data includes heartrate and heartrate variability sensor data generated from a heartrate sensor and accelerometer sensor data generated from an accelerometer sensor.

39. A computer program product comprising a computer readable storage medium having program code stored thereon, the program code executable by a processor of a mobile device to perform operations comprising:
maintaining, in a memory of the mobile device, a log of phone calls conducted using the mobile device over a window of time including a plurality of time periods, wherein each time period is a 24 hour time period;
determining an amount of time a user of the mobile device interacts with other persons on a per time period basis based on the log of phone calls;
comparing the amount of time the user of the mobile device interacts with the other persons on the per time period basis with a baseline amount of time;
for each time period in which the amount of time the user of the mobile device speaks with the other persons does not exceed the baseline amount of time, generating, at most once during the time period, a graphical user interface (GUI) rendered by a display screen of the mobile device, the GUI including a question set inquiring about a current mood of the user and providing a binary response option for each question of the question set;
for each time period the question set is presented via the GUI, receiving, via the GUI, a user input selecting a binary response option for each question of the question set, wherein the processor stores the binary response option selected in the memory of the mobile device for at least the window of time;
for each question of the question set presented by the GUI, selecting an expression from a plurality of expressions stored in the memory, the selecting performed based on a number of the time periods an affirmative response is received for the question as the binary response option;
determining a total score for the window of time by calculating, for each question of the question set presented, a per question score for the window of time using the selected expression for each respective question based on the affirmative responses received for the question and summing the per question scores;
determining whether the total score exceeds a threshold score; and
in response to determining that the total score exceeds the threshold score, transmitting, via a wireless transmitter of the mobile device, an electronic notification to a remote system of a health care provider, wherein the electronic notification indicates a need to follow-up with the user.

40. The computer program product of claim 39, wherein the program code is executable by the processor to perform operations further comprising:
periodically sampling audio detected via a microphone and an audio subsystem of the mobile device;
performing voice recognition on the sampled audio using the processor to detect whether a user of the mobile device is involved in a face-to-face conversation during the periodically sampling throughout the window of time; and
wherein the amount of time the user of the mobile device interacts with other persons is determined based on the log of phone calls and whether the user was determined to be involved in the face-to-face conversation from the periodically sampling.
